(19) United States
(12) Patent Application Publication
(10) Pub. No.: US 2026/0018253 A1
(43) Pub. Date: Jan. 15, 2026

(54) NON-TRANSITORY COMPUTER-READABLE RECORDING MEDIUM, ACCURACY PREDICTION METHOD, AND INFORMATION PROCESSING APPARATUS

(71) Applicant: Fujitsu Limited, Kawasaki-shi (JP)

(72) Inventor: Kazuhito MATSUDA, Kawasaki (JP)

(73) Assignee: Fujitsu Limited, Kawasaki-shi (JP)

(21) Appl. No.: 19/231,662

(22) Filed: Jun. 9, 2025

(30) Foreign Application Priority Data
Jul. 10, 2024 (JP) 2024-111239

(51) Int. Cl. G16C 10/00; G16C 20/70

(52) U.S. Cl. CPC G16C 10/00; G16C 20/70

(57) ABSTRACT

An information processing apparatus generates a regression equation for predicting the accuracy of computing the potential energy of a first molecule using each of a plurality of division patterns including a plurality of subsets including one or more atoms included in the first molecule. The information processing apparatus applies the regression equation to a plurality of division candidate patterns including a plurality of subsets including one or more atoms included in a second molecule. The information processing apparatus 10 then executes prediction of accuracy of computing the potential energy of the second molecule in the case of using each of the plurality of division candidates.

### CROSS-REFERENCE TO RELATED APPLICATION

This application is based upon and claims the benefit of priority of the prior Japanese Patent Application No. 2024-111239, filed on Jul. 10, 2024, the entire contents of which are incorporated herein by reference.

### FIELD

The embodiments discussed herein are related to a computer-readable recording medium, an accuracy prediction method, and an information processing apparatus.

### BACKGROUND

Molecular properties can be identified by obtaining the energy of the target molecule. For example, a stable state of the molecular structure can be clarified from the ground-state energy of the target molecule, and an unstable state of the molecular structure can be clarified from the excited-state energy of the target molecule.

Identification of molecular properties in such a manner is useful for drug discovery, discovery of new materials, and the like. Thus, quantum chemical computation is highly significant. Examples of the quantum chemical computation include coupled-cluster singles-and-doubles (-and-Triple) (CCSD (T)) as a classical algorithm, and variational quantum eigensolver (VQE) as a quantum algorithm assumed to be executed on a quantum computer.

The computational complexity of CCSD (T) may be O(n⁷), where n is the number of orbitals of a molecular. However, a current computer can compute only about 10¹ to 10². A similar degree of computational complexity is expected for VQE in the case of using a simulator. Also in the case of using noisy intermediate-scale quantum computer (NISQ), an increase in computational complexity in polynomial time is expected as the number of orbitals increases. This factor makes it unrealistic at present to apply an algorithm to the entire large molecule to obtain potential energy.

On the other hand, a known method uses a theory called the density matrix embedding theory (DMET) to divide an atomic group included in a molecule into several subsets, separately obtains the pieces of energy of the subsets, and then combines obtained pieces of energy to determine the entire potential energy. In the DMET, for example, when the energy of an alanine molecule is determined, the group of atoms included in alanine is divided into subsets, and the energy of each subset is calculated after abstracting the interaction with other subsets and then combined. This method can reduce the computational complexity (problem size). As described above, since the algorithm for obtaining the potential energy has a very large order of computational complexity, the computation time can be greatly reduced by using the DMET.
Patent Literature 1: U.S. Patent No. 2018/0,096,085

### SUMMARY

According to an aspect of an embodiment, a non-transitory computer-readable recording medium stores therein an accuracy prediction program that causes a computer to execute processing. The process includes generating a regression equation with which accuracy of computing potential energy of a first molecule is predicted by using each of a plurality of division patterns including a plurality of subsets each including one or more atoms included in the first molecule, and executing prediction of accuracy of computing potential energy of a second molecule in a case of using each of a plurality of division candidate patterns including a plurality of subsets each including one or more atoms included in the second molecule by applying the regression equation to the plurality of division candidate patterns.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating an information processing apparatus according to a first embodiment;

FIG. 2 is a diagram illustrating division patterns of a molecule;

FIG. 3 is a functional block diagram illustrating a functional configuration of the information processing apparatus according to the first embodiment;

FIG. 4 is a diagram illustrating a data structure used in the first embodiment;

FIG. 5 is a diagram illustrating a data structure used in the first embodiment;

FIG. 6 is a diagram illustrating a data structure used in the first embodiment;

FIG. 7 is a flowchart illustrating a flow of processing to derive a regression equation;

FIG. 8 is a flowchart illustrating a flow of processing to present division candidates;

FIG. 9 is a diagram illustrating a specific example of a division pattern;

FIG. 10 is a diagram illustrating a specific example of calculating energy accuracy;

FIG. 11 is a diagram illustrating a specific example of regression analysis;

FIG. 12 is a diagram illustrating a specific example of division candidates of a molecule to be calculated;

FIG. 13 is a diagram illustrating a specific example of calculating the estimated energy of a division candidate;

FIG. 14 is a diagram illustrating a specific example of creating the ranking of division candidates;

FIG. 15 is a diagram illustrating an example of a screen for presenting division candidates; and

FIG. 16 is a diagram illustrating a hardware configuration example.

### DESCRIPTION OF EMBODIMENTS

However, in the technique of division into subsets, the manner of division may produce the difference in the accuracy of the obtained energy of each subset, and the accuracy of computing potential energy may be deteriorated.

For example, an infinite number of patterns of molecule division exists even if constraints are imposed on the problem scale (e.g., the number of orbitals) of each subset, and the manner of division may produce the difference in the final accuracy of computing energy. This makes it unrealistic to randomly search for what division is preferable from an infinite number of candidates.

Preferred embodiments will be explained with reference to accompanying drawings. Note that the present invention is not limited by the embodiments. In addition, the embodiments can be appropriately combined within a range in which no conflict occurs.

### (a) First Embodiment

### Description of Information Processing Apparatus

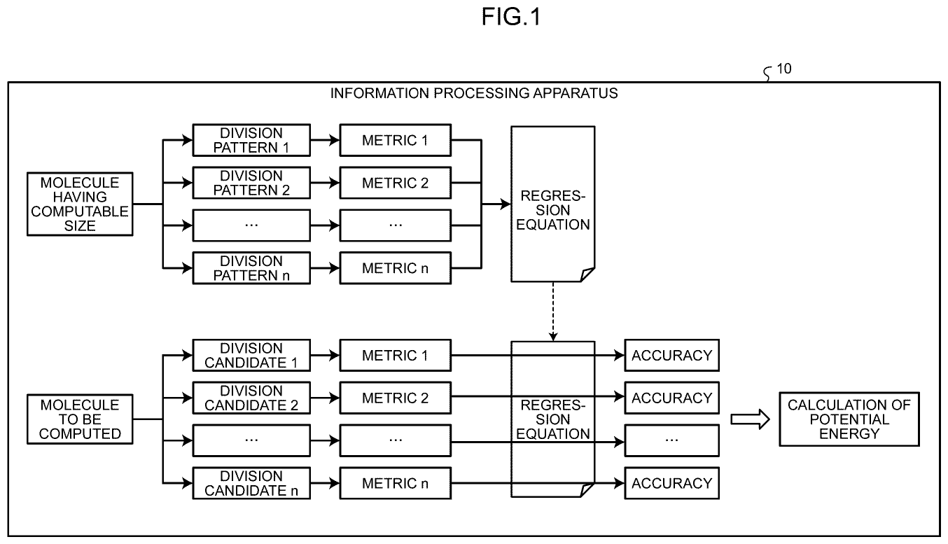

FIG. 1 is a diagram illustrating an information processing apparatus 10 according to a first embodiment. The information processing apparatus 10 illustrated in FIG. 1 is an example of a computer that divides an atom group included in a molecule into some subsets by using the theory called DMET, individually obtains the energy of each subset, and then combines the pieces of energy to calculate potential energy (hereinafter, it may be simply described as “energy”) of the whole (molecule).

Although the computational complexity of the potential energy of the molecule can be greatly reduced expected by using the DMET, the manner of division may produce the difference in the accuracy of the finally calculated potential energy. There are many patterns for dividing molecules even if constraints such as the number of orbitals are imposed. This requires the search for what kind of division is preferred.

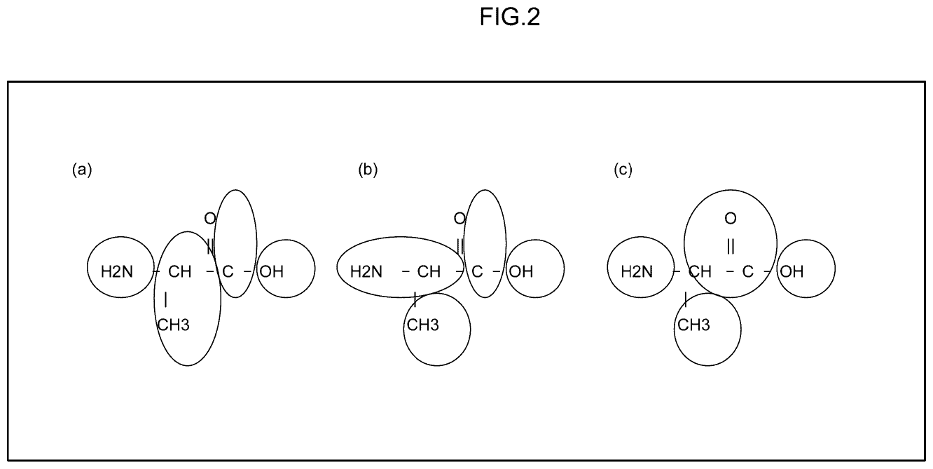

FIG. 2 is a diagram for illustrating division patterns of a molecule. FIG. 2 illustrates three division patterns (a), (b), and (c) as the division patterns of alanine. It takes a lot of time to calculate potential energy using each of countless division patterns, which is unrealistic. On the other hand, it is also conceivable to randomly narrow down countless division pattern candidates into the above three division patterns (a), (b), and (c) and calculate the accuracy of potential energy with the narrowed division patterns. However, since there is no criterion for narrowing down the candidates, the accuracy of the potential energy may decrease as a result of narrowing down the candidates. Hence, a method of randomly narrowing down the candidates is far from a realistic method.

In view of the above, the information processing apparatus 10 according to the first embodiment generates a regression equation for predicting the accuracy of computing the potential energy of a first molecule using each of a plurality of division patterns including a plurality of subsets including one or more atoms included in the first molecule. Subsequently, the information processing apparatus 10 applies the regression equation to a plurality of division candidate patterns (hereinafter, it may be referred to as division candidates) including a plurality of subsets including one or more atoms included in a second molecule. The information processing apparatus 10 then executes prediction of the accuracy of computing the potential energy of the second molecule in the case of using each of the plurality of division candidate patterns.

That is, when obtaining the energy of a large molecule using the DMET, the information processing apparatus 10 derives a regression equation for predicting the accuracy of computation on the basis of the number of orbitals, the number of electrons, and the like of each subset by using a molecule having such a size that the entire energy can be obtained (first molecule). Then, the information processing apparatus 10 divides a large molecule for which energy is desired to be obtained (second molecule) such that each subset falls within a computable size, and generates a plurality of division candidates. Thereafter, the information processing apparatus 10 applies the regression equation for predicting accuracy of computation to each of the plurality of generated division candidates, ranks the division candidates, and presents the ranked division candidates to the user.

For example, as illustrated in FIG. 1, the information processing apparatus 10 divides a molecule having a computable size into division patterns 1 to n (n is a natural number). Subsequently, the information processing apparatus 10 collects metrics 1 to n that are used as evaluation indices of the model in the regression analysis and are measurement criteria for the division patterns 1 to n, and calculates a regression equation using these metrics.

Thereafter, the information processing apparatus 10 generates division candidates 1 to n obtained by dividing a molecule to be computed for which energy is desired to be obtained under the same constraint as that in the division of a molecule having a computable size. Subsequently, the information processing apparatus 10 collects the metrics used in the generation of the regression equation (regression analysis) for each of the division candidates 1 to n, applies the collected metrics to the regression equation, and predicts the accuracy of computing of the energy calculation system. Thereafter, the information processing apparatus 10 calculates the potential energy of the molecule to be computed by using, for example, the division candidate 2 for which the best accuracy is ensured and executing the DMET or the like.

In this manner, the information processing apparatus 10 can predict division candidates for which the potential energy is computed with high accuracy by applying the regression equation generated using a molecule for which accurate potential energy can be calculated also to a molecule to be computed having a large size.

### Functional Configuration

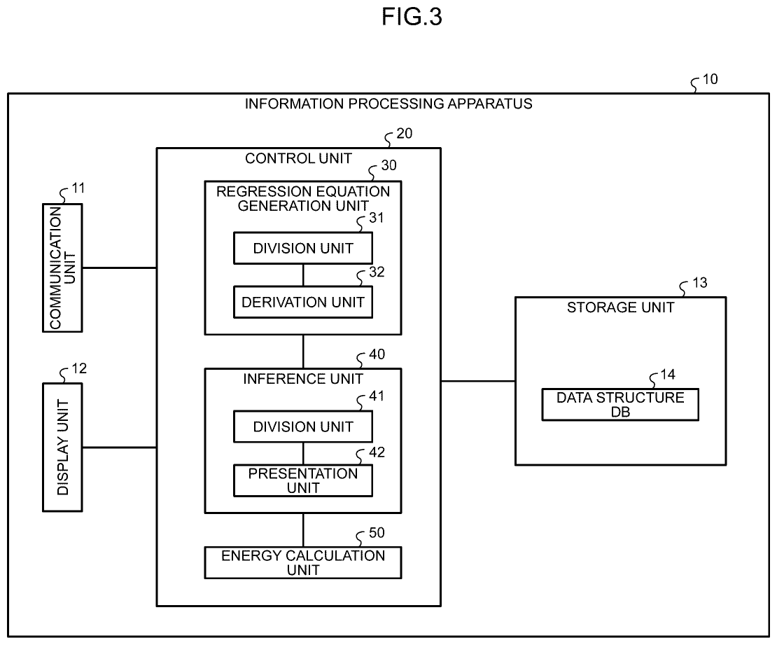

FIG. 3 is a functional block diagram illustrating a functional configuration of the information processing apparatus 10 according to the first embodiment. As illustrated in FIG. 3, the information processing apparatus 10 includes a communication unit 11, a display unit 12, a storage unit 13, and a control unit 20.

The communication unit 11 is a processing unit that controls communication with other devices, and is implemented by, for example, a communication interface. For example, the communication unit 11 receives, from a user terminal, an input of a molecule to be computed or the like for which energy is desired to be obtained. The communication unit 11 can also transmit various types of information calculated by the control unit 20 to the user terminal.

The display unit 12 is a processing unit that displays and outputs various types of information, and is implemented by, for example, a display or a touch panel. For example, the display unit 12 displays and outputs various types of information received by the communication unit 11 and various types of information calculated by the control unit 20.

The storage unit 13 is a processing unit that stores various data, programs executed by the control unit 20, and the like, and is implemented by, for example, a memory or a hard disk. For example, the storage unit 13 stores a data structure data base (DB) 14 including data used by the control unit 20 for various processes.

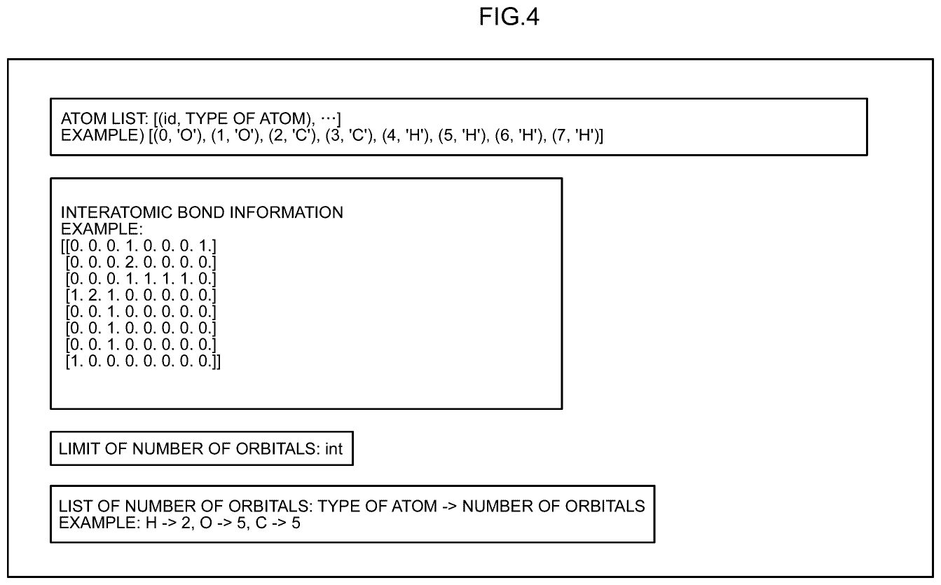

Here, a data structure of various types of information stored in the data structure DB 14 will be described. FIGS. 4, 5, and 6 are diagrams illustrating the data structure used in the first embodiment. As illustrated in FIG. 4, the data structure DB 14 includes data structures of an atom list, interatomic bond information, a limit of the number of orbitals, and a list of the number of orbitals.

Specifically, the term “atom list” refers to a list of atoms included in a molecule, and is represented by, for example, (id, type of atom). For example, (0, ‘O’) indicates that the atom of id=0 is “O”. The term “interatomic bond information” refers to bond information between atoms included in the molecule. When the value at a row number i and a column number j is n, the interatomic bond information is expressed as a symmetric matrix indicating that the i-th atom is n-tuple bonded to the j-th atom. The term “limit of the number of orbitals” refers to a threshold value (upper limit value) of the number of orbitals expressed by an integer data type (int), and for example, 8 is set. The term “list of the number of orbitals” refers to information defining the number of orbitals of each atom, and is expressed as “atom type→number of orbitals”. For example, “H→2” defines the fact that the number of orbitals of hydrogen “H” is “2”.

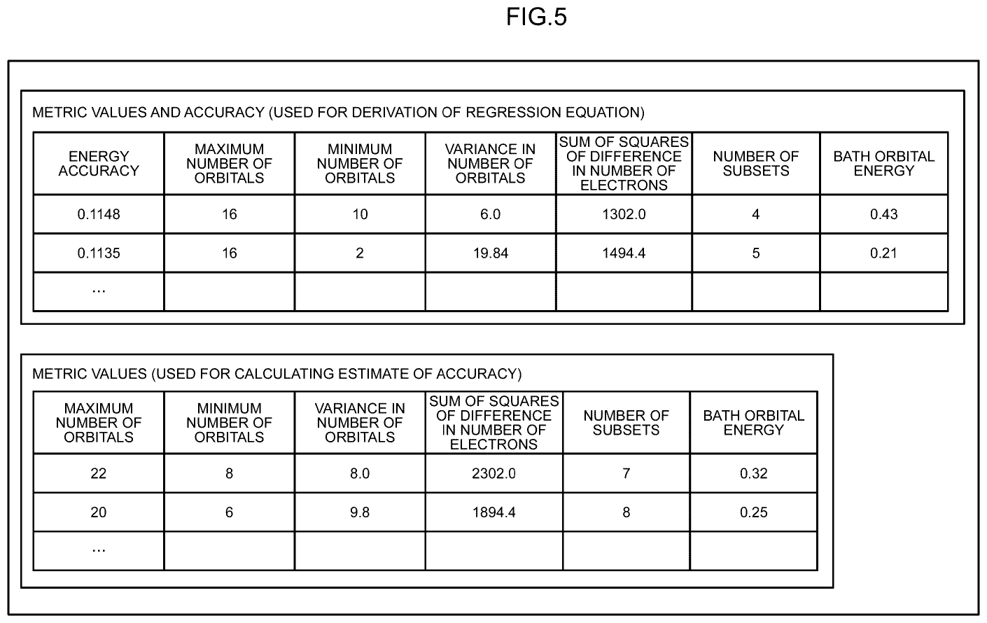

Subsequently, as illustrated in FIG. 5, the data structure DB 14 includes data structures of “metric values and accuracy” for division patterns obtained by dividing a computable molecule and “metric values” for division candidates obtained by dividing a molecule to be computed.

The term “metric values and accuracy” refers to information in which “accuracy, maximum number of orbitals, minimum number of orbitals, variance in the number of orbitals, sum of squares of the difference in the number of electrons, number of subsets, and bath orbital energy” are associated with each other. Here, the term “accuracy” refers to a difference between an accurate energy value obtained by CCSD (T) and an energy value calculated by the DMET or the like using the subset included in the division pattern. The term “maximum number of orbitals” refers to a maximum value of the number of orbitals of the subset included in the division pattern, and a value including spin is set. The “minimum number of orbitals” is a minimum value of the number of orbitals of the subset included in the division pattern, and a value including spin is set. The term “variance in the number of orbitals” refers to a variance value of the number of orbitals of each subset included in the division pattern. The term “sum of squares of the difference in the number of electrons” refers to the sum of squares of the difference between the total number of active electrons and the number of active atoms in each subset. The term “number of subsets” refers to the number of subsets included in the division pattern. The term “bath orbital energy” refers to the energy of a bath orbital that expresses an interaction between subsets in the DMET. The example of FIG. 5 illustrates that a certain division pattern of the computable molecule has “the energy accuracy of 0.1148, the maximum number of orbitals of 16, the minimum number of orbitals of 10, the variance in the number of orbitals of 6.0, the sum of squares of the difference in the number of electrons of 1302.0, the number of subsets of 4, and the bath orbital energy of 0.43”.

The term “metric values” refers to information about division candidates obtained by dividing the molecule to be computed and refers to “maximum number of orbitals, minimum number of orbitals, variance in the number of orbitals, sum of squares of the difference in the number of electrons, number of subsets, and bath orbital energy”. Each piece of information is the same as the content described above, and thus a detailed description thereof is omitted. The example of FIG. 5 illustrates that a certain division candidate of the molecule to be computed has “the maximum number of orbitals of 22, the minimum number of orbitals of 8, the variance in the number of orbitals of 8.0, the sum of squares of the difference in the number of electrons of 2302.0, the number of subsets of 7, and the bath orbital energy of 0.32”.

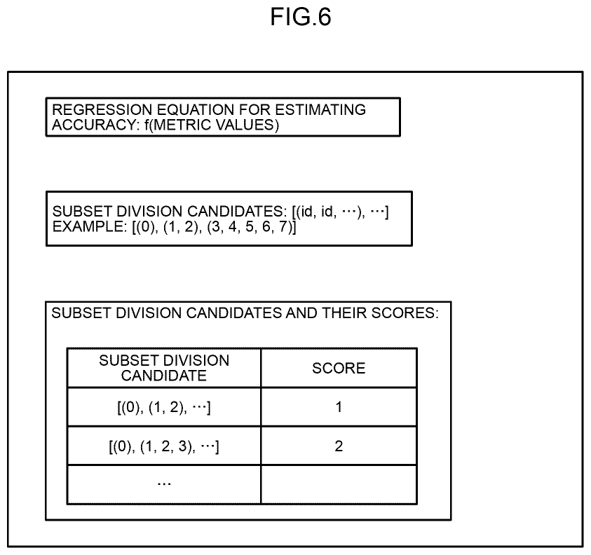

Furthermore, as illustrated in FIG. 6, the data structure DB 14 includes data structures of “regression equation for estimating accuracy”, “subset division candidates”, and “subset division candidates and their scores”.

The term “regression equation for estimating accuracy” refers to a regression equation for calculating the accuracy of energy. The regression equation is generated by the regression analysis by the control unit 20 and is “f (metric values)”. For example, the estimated energy accuracy is expressed by a linear combination of “c0×sum of squares of difference in number of electrons+c1×maximum number of orbitals+c2×minimum number of orbitals+c3×variance in number of orbitals+c4×bath orbital energy+c5×number of subsets+constant term”. Note that cX is a coefficient for each metric (X is 0 to 5 in this example).

The term “subset division candidates” refers to division candidates obtained by dividing the molecule to be computed, and a subset division candidate is represented by, for example, “(id, id . . . )”. For example, “(0), (1, 2) . . . ” indicates that the molecule is divided into the “subset consisting of an atom ‘O’”, where the id of the atom “O” is “0”, and the “subset including the atom ‘O’ whose id is ‘1’ and the atom ‘C’ whose id is ‘2’”.

The term “subset division candidates and their scores” refers to a ranking based on the accuracy of energy predicted for the subset division candidates by using a regression equation. The energy accuracy means a difference from the accurate energy obtained by the CCSD (T) for the subsets included in each division candidate. Therefore, the smaller the value of the energy accuracy, the higher the score. The example of FIG. 6 illustrates that the score “1” is calculated for the division candidate “(0), (1, 2) . . . ”.

The control unit 20 is a processing unit that controls the entire information processing apparatus 10, and is implemented by, for example, a processor. The control unit 20 includes a regression equation generation unit 30, an inference unit 40, and an energy calculation unit 50. The regression equation generation unit 30, the inference unit 40, and the energy calculation unit 50 are implemented by, for example, an electronic circuit included in a processor, or a process executed by the processor.

The regression equation generation unit 30 is a processing unit that includes a division unit 31 and a derivation unit 32 and generates a regression equation for predicting the accuracy of potential energy using the first molecule having a computable size.

The division unit 31 is a processing unit that divides the first molecule into a plurality of patterns including a plurality of subsets in which atoms included in the first molecule are bonded to each other. Specifically, the division unit 31 divides the first molecule into a plurality of patterns so that the total number of orbitals obtained by summing the number of orbitals of each atom included in the subset falls within the computable number of orbitals designated by the user or the like. For example, the division unit 31 can generate a plurality of candidates by executing the breadth-first search to construct a subset from atoms bonded to only one other atom and moving some atoms between subsets based on the constructed subset.

The division unit 31 can further generate a plurality of division patterns from a pattern having the largest number of orbitals not greater than the upper limit value (specific pattern). For example, the division unit 31 generates a plurality of division patterns from the specific pattern by moving an atom in each subset included in the specific pattern to another subset within a range in which the total number of orbitals is not greater than the upper limit value. At this time, for example, a constraint that one atom in each subset is to be moved can be imposed.

The derivation unit 32 is a processing unit that derives a regression equation for predicting the accuracy of computation on the basis of the number of orbitals, the number of electrons, and the like of each subset by using the first molecule having such a size that the entire energy can be obtained.

Specifically, the derivation unit 32 derives a regression equation by executing the following regression analysis on each of the plurality of patterns (division patterns). For example, the derivation unit 32 calculates the accurate potential energy of the first molecule by using CCSD (T) or the like and calculates estimated potential energy that is potential energy calculated from each pattern by using the DMET or the like. The derivation unit 32 then calculates a difference between the accurate potential energy of the first molecule and each estimated potential energy (energy accuracy) and calculates each metric value illustrated in FIG. 5. Thereafter, the derivation unit 32 executes regression analysis using the energy accuracy and each metric value, and generates a regression equation for estimating the energy accuracy.

The regression equation is expressed as a coefficient for each metric and a constant term as described above. Derivation of a regression equation involves normalization, and prediction of energy accuracy involves inverse transformation.

The inference unit 40 is a processing unit that includes a division unit 41 and a presentation unit 42 and uses the regression equation generated by the regression equation generation unit 30 to infer division candidates for calculating the energy of the second molecule to be computed.

The division unit 41 is a processing unit that generates a plurality of division candidates including a plurality of subsets in which atoms included in the second molecule are bonded to each other. Specifically, the division unit 41 generates a plurality of division candidates including a plurality of subsets from the second molecule by using a method the same as or similar to the method used by the division unit 31 at the time of derivation of the regression equation. That is, the division unit 41 generates a plurality of division candidates from the second molecule by using breadth-first search so as to be not greater than the upper limit value used at the time of generating the plurality of division patterns of the first molecule.

The presentation unit 42 is a processing unit that applies the regression equation to a plurality of division candidates including a plurality of subsets and generated from the second molecule and executes prediction of the accuracy of computing the potential energy of the second molecule in the case of using each of the plurality of division candidates. Furthermore, the presentation unit 42 is a processing unit that outputs information in which each of the plurality of division candidates is associated with the prediction result of the accuracy of computing the potential energy of the second molecule in a case of using a corresponding one of the plurality of division candidates.

The energy calculation unit 50 is a processing unit that calculates the potential energy of the second molecule. Specifically, the energy calculation unit 50 calculates the potential energy of the second molecule by the DMET using a division candidate for which the best accuracy of computation inferred (predicted) by the presentation unit 42 is ensured. For example, the energy calculation unit 50 calculates the potential energy of the second molecule by calculating the energy of each of the plurality of subsets included in the division candidate for which the highest accuracy of computation is ensured, and then combining the pieces of energy of each of the plurality of subsets. A quantum simulator or the like can also be used for the bonding computation.

### Flow of Derivation Processing of Regression Equation

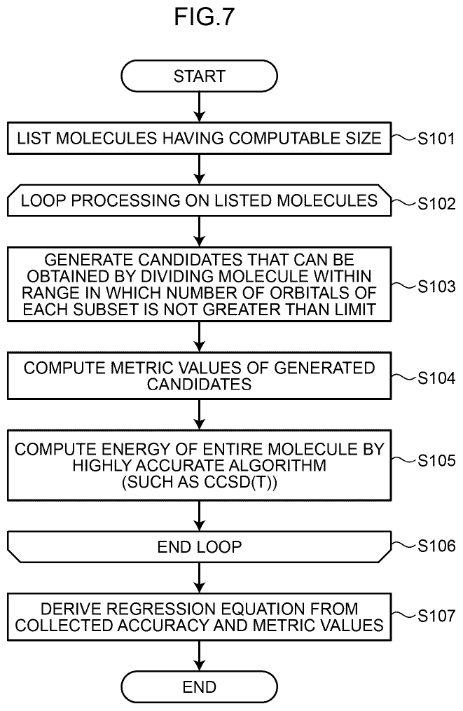

FIG. 7 is a flowchart illustrating a flow of processing to derive a regression equation. As illustrated in FIG. 7, the regression equation generation unit 30 lists molecules having a computable size (S101), and loops the following processing for the listed molecules (S102 to S106).

Specifically, the regression equation generation unit 30 generates candidates that can be obtained by dividing the molecule within a range in which the number of orbitals of each subset is not greater than the limit (not greater than the upper limit value) (S103), computes metric values of the generated candidates (S104), and computes the energy of the entire molecule by a highly accurate algorithm, for example, CCSD (T) (S105). Note that S103 to S105 may be executed in parallel for each molecule.

When the loop processing ends (S106), the regression equation generation unit 30 derives a regression equation from the collected accuracy and metric values (S107).

### Flow of Processing to Present Division Candidates

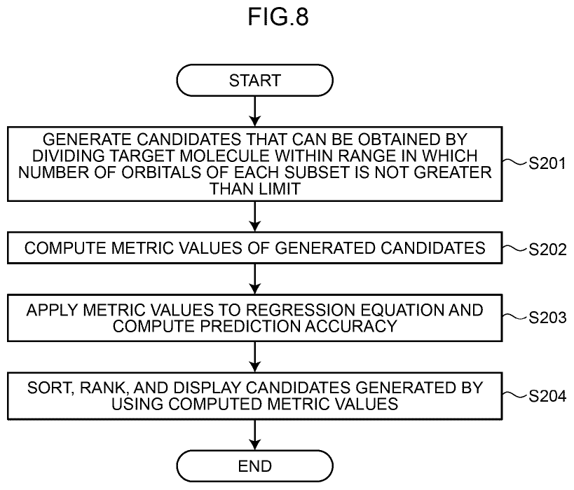

FIG. 8 is a flowchart illustrating a flow of processing to present division candidates. As illustrated in FIG. 8, the inference unit 40 generates division candidates that can be obtained by dividing the target molecule within a range in which the number of orbitals of each subset is not greater than the limit (S201).

Subsequently, the inference unit 40 computes the metric values of the generated division candidates (S202), and computes the prediction accuracy by applying the metric values to the regression equation (S203). Thereafter, the inference unit 40 sorts, ranks, and displays the generated division candidates on the basis of the computed metric values (S204).

### Specific Example

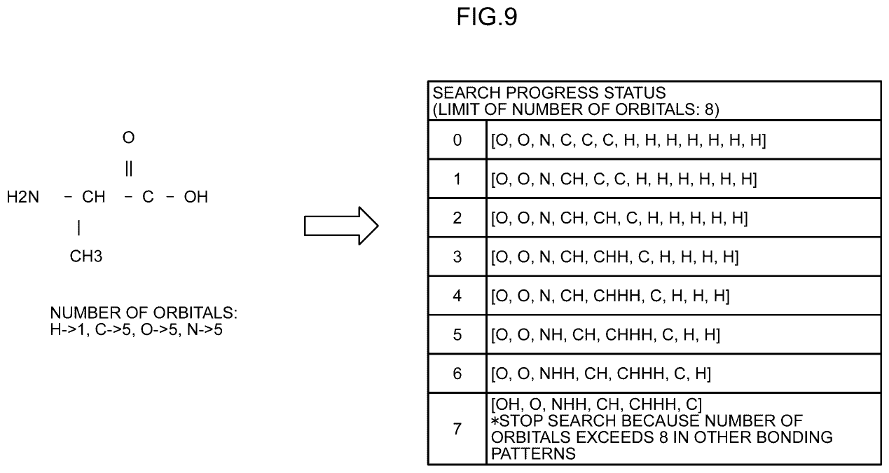

Next, specific examples of the generation of a regression equation using the above-described first molecule and the calculation of the potential energy of the second molecule will be described with reference to FIGS. 9 to 15. Here, alanine (C₃H₇NO₂) is used as an example of the computable first molecule, and heptanoic acid (C₇H₁₄O₂) is used as an example of the second molecule to be computed.

### Derivation of Regression Equation: Generation of Division Patterns

First, the information processing apparatus 10 breaks down alanine into a plurality of division patterns with the computable number of orbitals as an upper limit of the total number of orbitals of each subset. FIG. 9 is a diagram illustrating a specific example of a division pattern. As illustrated in FIG. 9, the information processing apparatus 10 generates a pattern obtained by dividing atoms included in alanine (C₃H₇NO₂) to a plurality of subsets by breadth-first search in consideration of the connection in the molecular structure under the constraint that the number of orbitals is not greater than the limit of the number of orbitals of “8”. Here, a breadth-first search starting from an atom connected to only one other atom is used.

For example, the information processing apparatus 10 generates a pattern “O, O, N, C, C, C, H, H, H, H, H, H, H” with each atom as a subset. The maximum number of orbitals in this pattern is “N=5, O=5, and C=5” and is not greater than the limit value of the number of orbitals (8), and accordingly this pattern is adopted as the search result (ID=0).

From the pattern of “ID=0”, the information processing apparatus 10 then generates a pattern “O, O, N, CH, C, C, H, H, H, H, H, H” in which “C” and the adjacent “H” are bonded to each other in accordance with the molecular structure. The maximum number of orbitals in this pattern is “CH=5+1=6” and is not greater than the limit value of the number of orbitals (8), and accordingly this pattern is adopted as the search result (ID=1).

From the pattern of “ID=1”, the information processing apparatus 10 further generates a pattern “O, O, N, CH, CH, C, H, H, H, H, H” in which “C” and the adjacent “H” are bonded to each other in accordance with the molecular structure. The maximum number of orbitals in this pattern is “CH=5+1=6” and is not greater than the limit value of the number of orbitals (8), and accordingly this pattern is adopted as the search result (ID=2).

From the pattern of “ID=2”, the information processing apparatus 10 then generates a pattern “O, O, N, CH, CHH, C, H, H, H, H” in which “CH” and the adjacent “H” are bonded to each other in accordance with the molecular structure. The maximum number of orbitals in this pattern is “CHH=5+1+1=7” and is not greater than the limit value of the number of orbitals (8), and accordingly this pattern is adopted as the search result (ID=3).

From the pattern of “ID=3”, the information processing apparatus 10 further generates a pattern “O, O, N, CH, CHHH, C, H, H, H” in which “CHH” and the adjacent “H” are bonded to each other in accordance with the molecular structure. The maximum number of orbitals in this pattern is “CHHH=5+1+1+1=8” and is not greater than the limit value of the number of orbitals (8), and accordingly this pattern is adopted as the search result (ID=4).

From the pattern of “ID=4”, the information processing apparatus 10 then generates a pattern “O, O, NH, CH, CHHH, C, H, H” in which “N” and the adjacent “H” are bonded to each other in accordance with the molecular structure. The maximum number of orbitals in this pattern is “CHHH=5+1+1+1=8” and is not greater than the limit value of the number of orbitals (8), and accordingly this pattern is adopted as the search result (ID=5).

From the pattern of “ID=5”, the information processing apparatus 10 further generates a pattern “O, O, NHH, CH, CHHH, C, H” in which “NH” and the adjacent “H” are bonded to each other in accordance with the molecular structure. The maximum number of orbitals in this pattern is “CHHH=5+1+1+1=8” and is not greater than the limit value of the number of orbitals (8), and accordingly this pattern is adopted as the search result (ID=6).

From the pattern of “ID=6”, the information processing apparatus 10 further generates a pattern “OH, O, NHH, CH, CHHH, C” in which “O” and the adjacent “H” are bonded to each other in accordance with the molecular structure. The maximum number of orbitals in this pattern is “CHHH=5+1+1+1=8” and is not greater than the limit value of the number of orbitals (8), and accordingly this pattern is adopted as the search result (ID=7).

In a case in which adjacent atoms are thereafter combined to each other from the pattern of ID=7 “OH, O, NHH, CH, CHHH, C”, the potential subset would include “CO” and “CH—CHHH”, and the maximum number of orbitals exceeds the limit value of the number of orbitals (8). Therefore, the information processing apparatus 10 ends the division. As a result, the information processing apparatus 10 generates eight patterns having ID=0 to 7.

### Derivation of Regression Equation: Calculation of Energy Accuracy

Next, the information processing apparatus 10 further generates a plurality of candidates from the obtained subsets. Here, one atom is moved from each subset, and if applicable, the subset in which the number of orbitals exceeds the limit is further divided.

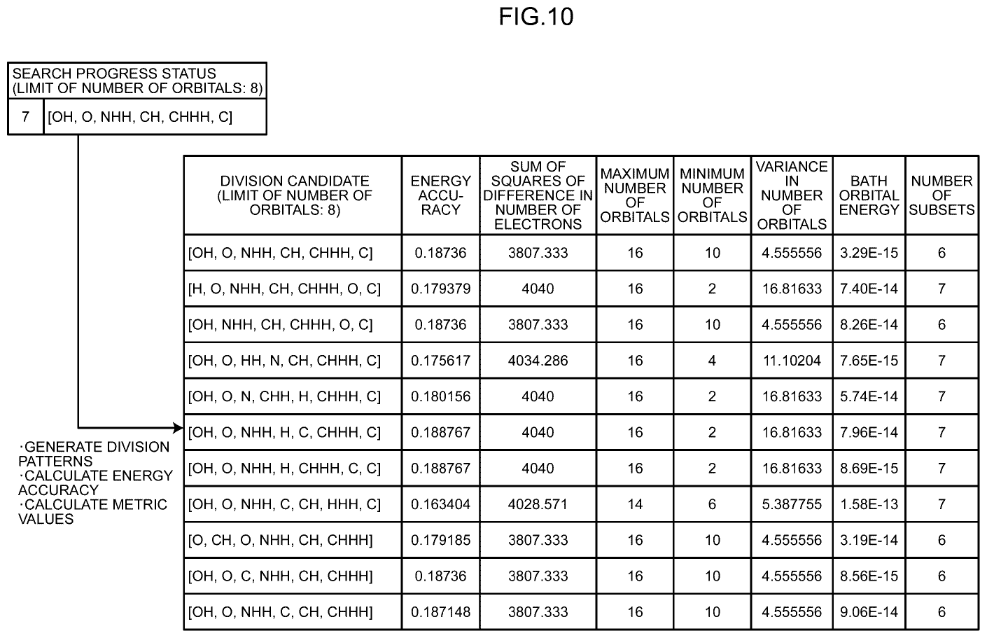

FIG. 10 is a diagram illustrating a specific example of calculating energy accuracy. As illustrated in FIG. 10, the information processing apparatus 10 adopts, as a division pattern, a pattern having the largest number of orbitals from the patterns generated in FIG. 9. In this example, the information processing apparatus 10 selects the pattern of ID=7 “OH, O, NHH, CH, CHHH, C” having the largest number of orbitals and the smallest number of divided subsets. Note that the smaller number of divided subsets is more likely to lead to further generation of a division pattern.

Then, the information processing apparatus 10 executes the breadth-first search described with reference to FIG. 9 on the pattern of ID=7 “OH, O, NHH, CH, CHHH, C” and generates a plurality of division patterns. In the example of FIG. 10, the information processing apparatus 10 generates eleven division patterns such as division patterns “H, O, NHH, CH, CHHH, O, C” and “OH, NHH, CH, CHHH, O, C” from “OH, O, NHH, CH, CHHH, C” of ID=7.

The information processing apparatus 10 then collects metric values (energy accuracy, sum of squares of the difference in the number of electrons, maximum number of orbitals, minimum number of orbitals, variance in the number of orbitals, bath orbital energy, and number of subsets) for each of the eleven division patterns. Here, a division pattern “OH, O, NHH, CH, CHHH, C” will be described as an example.

For example, the information processing apparatus 10 calculates accurate the potential energy of alanine using the CCSD (T). The information processing apparatus 10 calculates estimated potential energy obtained from a division pattern by calculating the energy of each of the subsets “OH”, “O”, “NHH”, “CH”, “CHHH”, and “C” using the DMET, a quantum algorithm, or the like, and combining them. The information processing apparatus 10 then calculates a difference between accurate potential energy and estimated potential energy as “energy accuracy: 0.8736”.

The information processing apparatus 10 sets the result of calculating the sum of squares of the difference between the total number of active electrons and the number of active atoms in each subset as the “sum of squares of difference in number of electrons: 3807.333”. The information processing apparatus 10 sets “16” obtained by doubling the maximum number of orbitals “CHHH=8” of the division pattern in consideration of spin as “maximum number of orbitals”. The information processing apparatus 10 sets “10” obtained by doubling the minimum number of orbitals “C=5” of the division pattern in consideration of spin as “minimum number of orbitals”. The information processing apparatus 10 calculates a variance value from the number of orbitals including spin and sets “variance in number of orbitals: 4.555556”. The information processing apparatus 10 calculates the energy of the bath orbital using the DMET and sets “bath orbital energy: 3.29E-15”. The information processing apparatus 10 sets the number of subsets “6” of the division pattern “OH, O, NHH, CH, CHHH, C”.

The information processing apparatus 10 uses the above-described method and collects metric values (energy accuracy, sum of squares of the difference in the number of electrons, maximum number of orbitals, minimum number of orbitals, variance in the number of orbitals, bath orbital energy, and number of subsets) for each of the eleven division patterns generated from the pattern of ID=7 “OH, O, NHH, CH, CHHH, C”.

### Derivation of Regression Equation: Regression Analysis

Next, the information processing apparatus 10 generates a regression equation by using the metric values and the energy accuracy of each division candidate obtained in FIG. 10. For example, the information processing apparatus 10 generates a regression equation for calculating energy accuracy from the metric values by executing regression analysis with “energy accuracy” of each division candidate as a response variable and each metric value as an explanatory variable. That is, since the value of “energy accuracy” calculated by the regression equation is information indicating a difference from the accurate potential energy of alanine by using the CCSD (T), the smaller value indicates the better accuracy.

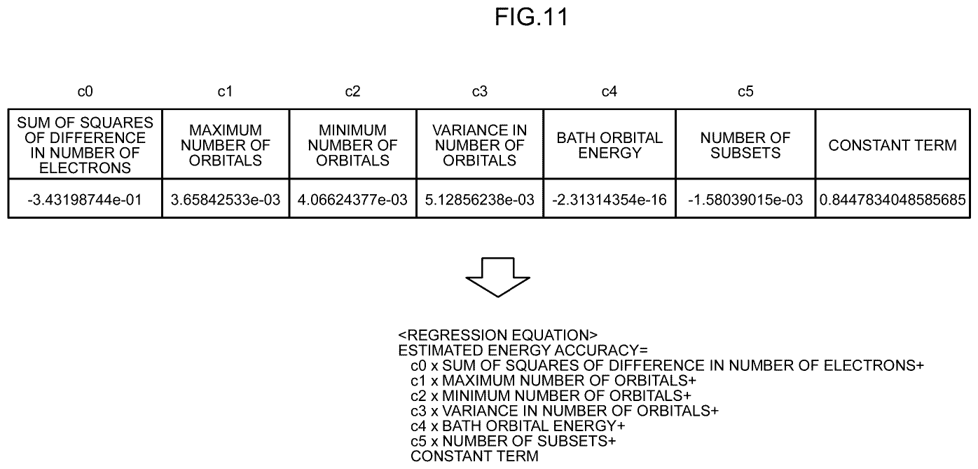

FIG. 11 is a diagram illustrating a specific example of regression analysis. As illustrated in FIG. 11, the information processing apparatus 10 generates, as regression equation for calculating estimated energy accuracy, a regression equation expressed by a linear combination of “c0×sum of squares of difference in number of electrons+c1×maximum number of orbitals+c2×minimum number of orbitals+c3×variance in number of orbitals+c4×bath orbital energy+c5×number of subsets+constant term”. Note that a numerical value corresponding to each metric illustrated in FIG. 11 is a coefficient such as c0 or c1, or a constant term. For example, the maximum number of orbitals “3.65842533×10⁻³” corresponds to the coefficient “c1”.

### Derivation of Potential Energy of Molecule: Generation of Division Candidates

When the generation of the regression equation is completed, the information processing apparatus 10 generates division patterns by dividing heptanoic acid (C₇H₁₄O₂) to be computed by a method the same as or similar to that at the time of generating the regression equation.

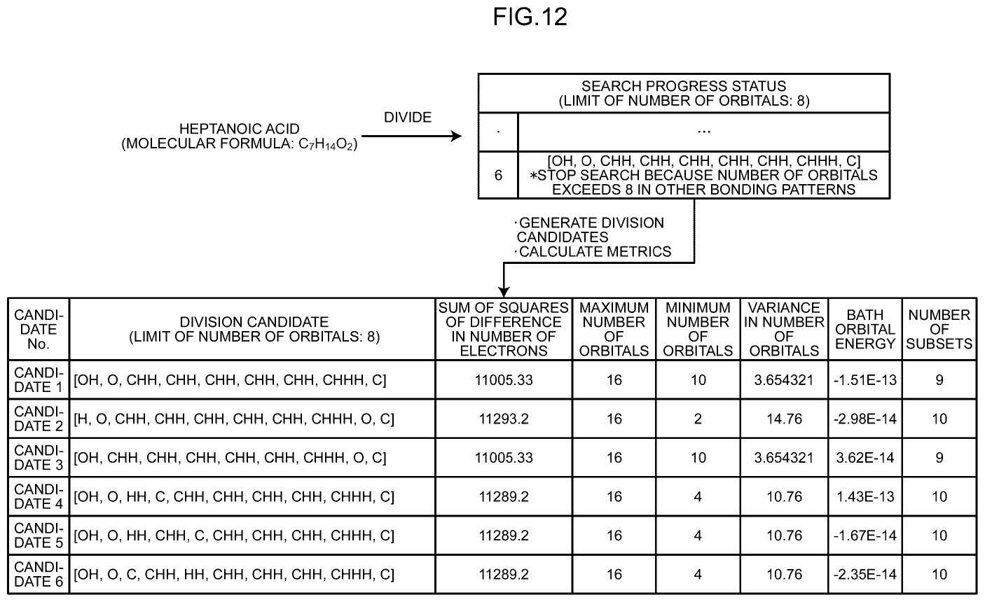

FIG. 12 is a diagram illustrating a specific example of the division candidates of a molecule to be calculated. As illustrated in FIG. 12, the information processing apparatus 10 generates a pattern obtained by dividing the molecule into a plurality of subsets by breadth-first search in consideration of the connection in the molecular structure so that the number of orbitals is not greater than the limit of the number of orbitals of “8”. The information processing apparatus 10 then identifies a division pattern of ID=6 “OH, O, CHH, CHH, CHH, CHH, CHH, CHHH, C” having the largest number of orbitals.

Thereafter, the information processing apparatus 10 moves one atom from each subset in the division pattern of ID=6 “OH, O, CHH, CHE, CHH, CHH, CHH, CHHH, C” and generates division candidates 1 to 6 in which the number of orbitals does not exceed the limit. Then, the information processing apparatus 10 calculates the metric values for each of the division candidates 1 to 6 by a method the same as or similar to the method described with reference to FIG. 10. As a result, the information processing apparatus 10 can collect the metric values of each division candidate.

### Derivation of Potential Energy of Molecule: Calculation of Estimated Energy

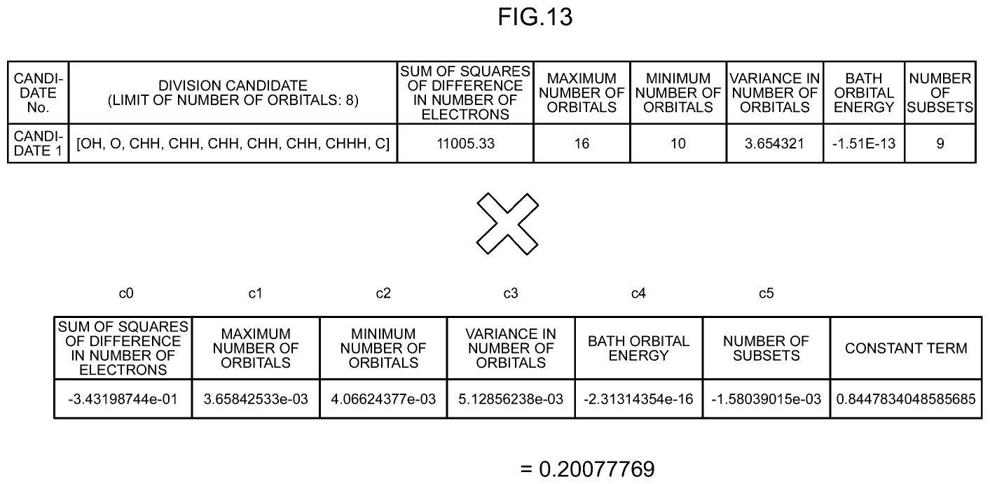

Next, the information processing apparatus 10 calculates energy accuracy for each of the division candidates 1 to 6. FIG. 13 is a diagram illustrating a specific example of calculating the estimated energy of the division candidate. As illustrated in FIG. 13, the information processing apparatus 10 multiplies each of the metric values (sum of squares of difference in number of electrons: 11005.33, maximum number of orbitals: 16, minimum number of orbitals: 10, variance in number of orbitals: 3.654321, bath orbital energy:−1.5×10⁻¹³, and number of subsets: 9) of the division candidate 1 by a corresponding one of coefficients c0 to c5 “sum of squares of difference in number of electrons (=c0), maximum number of orbitals (=c1), minimum number of orbitals (=c2), variance in number of orbitals (=c3), bath orbital energy (=c4), and number of subsets (=c5)” of the regression equation. As a result, the information processing apparatus 10 calculates a value “0.20077769” obtained by adding each product and a constant term as the energy accuracy.

As described above, since the energy accuracy calculated here is a value indicating a difference from the accurate potential energy, the smaller value indicates the better accuracy. Although FIG. 13 illustrates the division candidate 1 of FIG. 12, the same or similar operation is executed on the division candidates 2 to 6.

### Derivation of Potential Energy of Molecule: Ranking

Next, the information processing apparatus 10 calculates “energy accuracy” for each of the division candidates 1 to 6 by the method described with reference to FIG. 13, and ranks the division candidates 1 to 6 in descending order of “energy accuracy”.

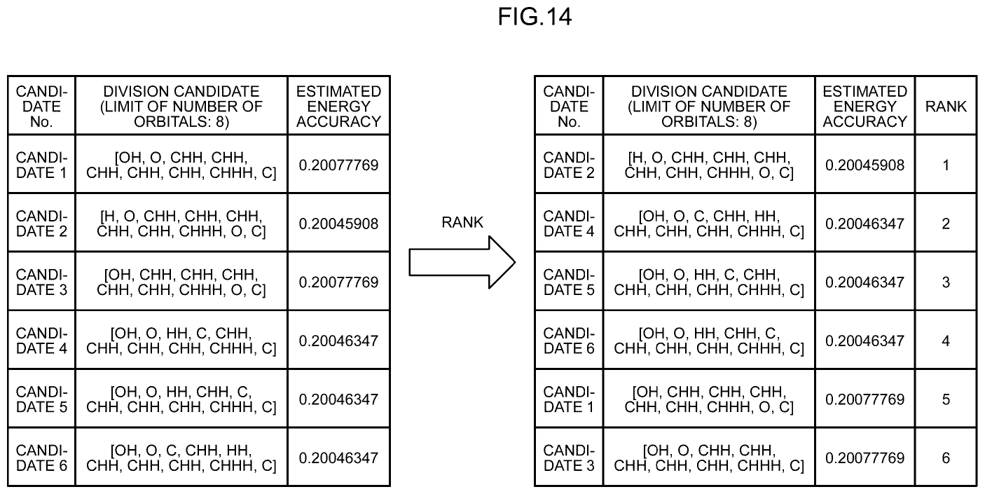

FIG. 14 is a diagram illustrating a specific example of creating the ranking of division candidates. As illustrated in FIG. 14, the information processing apparatus 10 calculates the estimated energy accuracy for each of the division candidates 1 to 6 described with reference to FIG. 12 by the method described with reference to FIG. 13. For example, the information processing apparatus 10 calculates “estimated energy accuracy: 0.20077769” for the division candidate 1, “estimated energy accuracy: 0.20045908” for the division candidate 2, and “estimated energy accuracy: 0.20077769” for the division candidate 3. Similarly, the information processing apparatus 10 calculates “estimated energy accuracy: 0.20046347” for the division candidate 4, “estimated energy accuracy: 0.20046347” for the division candidate 5, and “estimated energy accuracy: 0.20046347” for the division candidate 6.

Then, the information processing apparatus 10 executes ranking in descending order of estimated energy accuracy. Specifically, the information processing apparatus 10 executes ranking under specified conditions. Examples of specified conditions include: a condition in which a higher rank is assigned to a smaller value of the estimated energy accuracy; a condition in which a higher rank is assigned to the smaller number of subsets when the values of the estimated energy accuracy are the same; a condition in which a higher rank is assigned to the smaller candidate number, and a condition in which a higher rank is assigned to the greater bath orbital energy or the greater sum of squares of the difference in the number of electrons. In the example of FIG. 14, the information processing apparatus 10 executes ranking so as to place the candidates in the order of the division candidate 2, the division candidate 4, the division candidate 5, the division candidate 6, the division candidate 1, and the division candidate 3. The smaller the value, the higher the rank.

### Derivation of Potential Energy of Molecule: Presentation

Finally, the information processing apparatus 10 presents the information ranked in FIG. 14 to the user by outputting the information to the display unit 12 or transmitting the information to the user terminal.

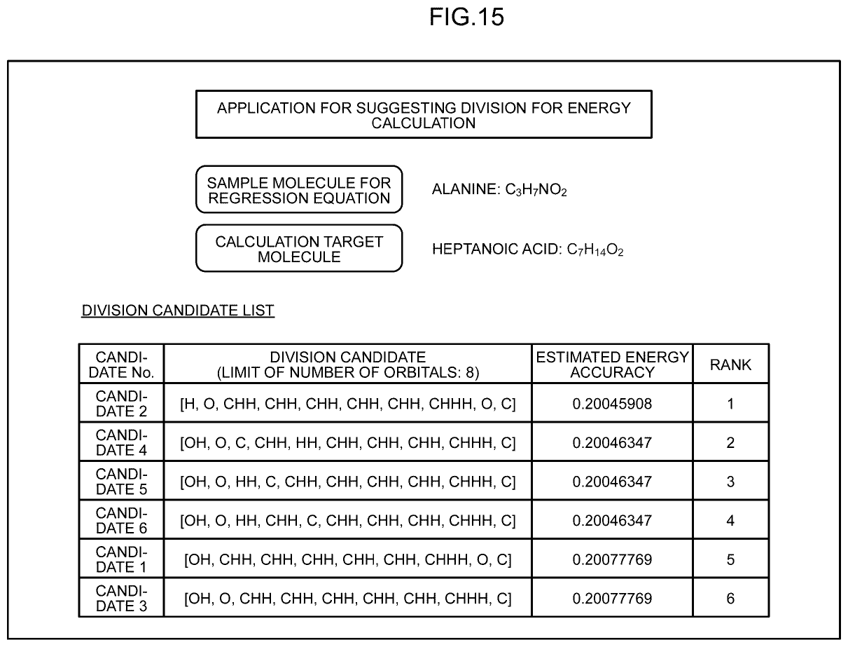

FIG. 15 is a diagram illustrating an example of a screen for presenting division candidates. As illustrated in FIG. 15, the information processing apparatus 10 outputs a screen that displays the ranking of the division candidates obtained in FIG. 14. For example, this screen includes: a “sample molecule for regression equation” indicating a molecule selected for generating a regression equation (e.g., alanine); a “calculation target molecule” indicating a molecule selected as a calculation target of potential energy (e.g., heptanoic acid); and a “division candidate list” indicating No of each division candidate and the subset information, the estimated energy accuracy, and the rank of each division candidate. As the information of the division candidate list, information obtained in the process on the way to the completion of ranking as illustrated in FIG. 14 is used. The information displayed on the screen is merely an example as long as at least a first ranked division candidate is displayed, and other information can be freely changed.

Thereafter, the information processing apparatus 10 calculates the potential energy of heptanoic acid by using the first ranked division candidate or a division candidate selected by the user from the division candidate list.

### Effects

As described above, the information processing apparatus 10 derives the regression equation for estimating the accuracy of the potential energy by using the molecule having such a size that the entire energy can be obtained, and calculates the estimation accuracy of the energy for division candidates for the large molecule for which the energy is desired to be obtained. As a result, the information processing apparatus 10 can predict division candidates for which the potential energy is computed with high accuracy.

The information processing apparatus 10 derives a regression equation that is a result of analyzing, by regression analysis, a relationship between metric values and estimated energy accuracy indicating a difference between energy actually calculated by the CCSD (T) or the like and estimated potential energy. The information processing apparatus 10 determines the energy accuracy of the division candidates of the target molecule using such a regression equation. Therefore, the information processing apparatus 10 can improve the accuracy of the finally obtained potential energy as compared to a case of randomly generating division candidates or adopting a division candidate designated by the user.

The information processing apparatus 10 can suppress endless generation of division candidates by imposing a constraint (e.g., the number of orbitals) when generating the division candidates. Therefore, the information processing apparatus 10 can suppress the prolongation of the flow of a series of processing from the generation of the regression equation to final energy calculation. As a result of being able to suppress prolongation, the information processing apparatus 10 can reduce the processing load on the processor of the information processing apparatus 10 and increase the processing speed.

The information processing apparatus 10 imposes the same constraint at the time of generating division candidates in deriving the regression equation and in calculating the estimated energy accuracy. Accordingly, the information processing apparatus 10 calculates the estimated energy accuracy under the same condition as the condition of the regression equation, and thus, the estimated energy accuracy can be calculated with high accuracy.

### (b) Second Embodiment

Although the embodiment of the present invention have been described so far, the present invention may be carried out in various different forms other than the above-described embodiment.

### Numerical Value or the Like

The numerical values, division method, and the like used in the above embodiment are merely examples, and can be freely changed. Each value is not necessarily accurate, and is merely an example. The flow of the processing described with reference to each flowchart can be appropriately changed within a range in which no conflict occurs.

### Metrics

The above embodiment has described the example in which “maximum number of orbitals, minimum number of orbitals, variance in number of orbitals, sum of squares of the difference in the number of electrons, number of subsets, and bath orbital energy” are used as the metrics. However, one or more of them may be lacked, and at least one or more of them can be used in combination.

### Division Method

The above embodiment has described the example in which the information processing apparatus 10 executes two-step division, but derivation of a regression equation and calculation of estimated energy accuracy can also be executed by one-step division. For example, the information processing apparatus 10 selects one pattern from among patterns including subsets at both the time of deriving the regression equation and the time of calculating the estimated energy accuracy, and further generates a division pattern from the selected one pattern. Then, the information processing apparatus 10 further divides the division pattern, derives the regression equation, and calculates the estimated energy accuracy. In the above embodiment, this example has been described, but the present invention is not limited thereto. That is, the information processing apparatus 10 can derive a regression equation by computing the metric values and the like at the step in FIG. 9 instead of that in FIG. 10.

### System

The processing procedure, the control procedure, the specific name, and the information including various data and parameters described in the document or illustrated in the drawings may be freely changed unless otherwise specified.

Specific forms of distribution and integration of the components of each unit or device are not limited to those illustrated in the drawings. For example, the regression equation generation unit 30 and the inference unit 40 may be integrated. That is, all or a part of the components may be functionally or physically distributed/integrated in any unit depending on various loads, usage conditions, and the like. All or any part of each processing function of the units and devices can be implemented by a central processing unit (CPU) and a program analyzed and executed by the CPU, or can be implemented as hardware based on wired logic.

Furthermore, all or any part of each processing function executed by the units and devices can be implemented by a CPU and a program analyzed and executed by the CPU, or can be implemented as hardware based on wired logic.

### Hardware

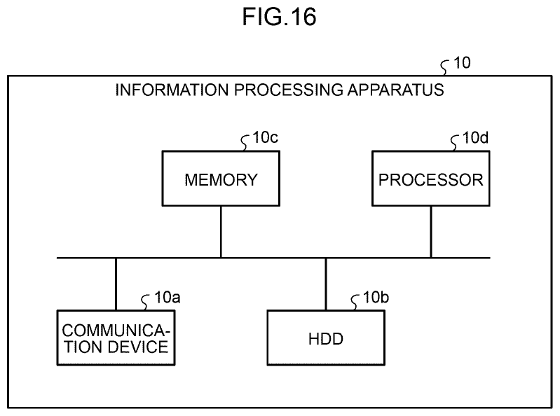

FIG. 16 is a diagram illustrating a hardware configuration example. As illustrated in FIG. 16, the information processing apparatus 10 includes a communication device 10a, a hard disk drive (HDD) 10b, a memory 10c, and a processor 10d. The components illustrated in FIG. 16 are connected to each other by a bus or the like.

The communication device 10a is a network interface card or the like and communicates with other devices. The HDD 10b stores programs for operating the functions illustrated in FIG. 3 and DBs.

The processor 10d runs the process of executing the functions with reference to FIG. 3 and the like by reading, from the HDD 10b or the like, a program for executing processing the same as or similar to the processing executed by each processing unit illustrated in FIG. 3, and developing the program in the memory 10c. For example, this process executes functions the same as or similar to those of the processing units included in the information processing apparatus 10. Specifically, the processor 10d reads, from the HDD 10b and the like, a program having functions the same as or similar to those of the regression equation generation unit 30, the inference unit 40, the energy calculation unit 50, and the like. Then, the processor 10d run the process of executing processing the same as or similar to the processing executed by the regression equation generation unit 30, the inference unit 40, the energy calculation unit 50, and the like.

In this manner, the information processing apparatus 10 operates as an information processing apparatus that executes the energy calculation method by reading and executing the program. Alternatively, the information processing apparatus 10 can also implement functions the same as or similar to those of the above-described embodiment by reading the program from the recording medium by the medium reading device and executing the read program. Note that the program referred to in the other embodiment is not limited to being executed by the information processing apparatus 10. For example, the above embodiment may be similarly applied to a case in which another computer or server executes a program or a case in which they execute a program in cooperation.

The program may be distributed via a network such as the Internet. In addition, the program may be recorded on a computer-readable recording medium such as a hard disk, a flexible disk (FD), a CD-ROM, a magneto-optical disk (MO), or a digital versatile disc (DVD), and may be executed by being read from the recording medium by the computer.

According to an embodiment, it is possible to predict division candidates for which the potential energy is computed with high accuracy.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiment(s) of the present invention has (have) been described in detail, it should be understood that the various changes, substitutions, and alterations could be made hereto without departing from the spirit and scope of the invention.

What is claimed is:

1. A non-transitory computer-readable recording medium having stored therein an accuracy prediction program that causes a computer to execute processing comprising:
generating a regression equation with which accuracy of computing potential energy of a first molecule is predicted by using each of a plurality of division patterns including a plurality of subsets each including one or more atoms included in the first molecule; and
executing prediction of accuracy of computing potential energy of a second molecule in a case of using each of a plurality of division candidate patterns including a plurality of subsets each including one or more atoms included in the second molecule by applying the regression equation to the plurality of division candidate patterns.

2. The non-transitory computer-readable recording medium according to claim 1, wherein
the generating includes
generating, for each of the plurality of division patterns, the regression equation by linear combination using at least one of the number of orbitals of each of the plurality of subsets included in the division pattern, the number of electrons of each of the plurality of subsets, the number of the plurality of subsets included in the division pattern, and energy of a bath orbital expressing an interaction between the plurality of subsets.

3. The non-transitory computer-readable recording medium according to claim 2, wherein
the generating includes
generating, for each of the plurality of division patterns, the regression equation by the linear combination using a maximum number of orbitals and a minimum number of orbitals of each of the plurality of subsets included in the division pattern and a variance value of the number of orbitals of each of the plurality of subsets when the regression equation is generated by the linear combination using the number of orbitals.

4. The non-transitory computer-readable recording medium according to claim 1, wherein
the generating includes
generating the plurality of division patterns from the first molecule, the plurality of division patterns having a total number of orbitals obtained by summing the number of orbitals of each atom included in the plurality of subsets is not greater than an upper limit value,
specifying a specific pattern having the total number of orbitals being largest among the plurality of division patterns,
generating a plurality of division candidate patterns from the specific pattern by moving an atom in each of the plurality of subsets included in the specific pattern to another subset among the plurality of subsets within a range in which the total number of orbitals is not greater than the upper limit value, and
generating the regression equation by using each of the plurality of division candidate patterns.

5. The non-transitory computer-readable recording medium according to claim 4, wherein
the executing includes
generating the plurality of division candidate patterns from the second molecule, the plurality of division candidate patterns having the total number of orbitals not greater than the upper limit value used at a time of generating the plurality of division patterns of the first molecule.

6. The non-transitory computer-readable recording medium according to claim 1, wherein the process further includes:
calculating energy of each of the plurality of subsets included in a division candidate pattern for which a highest accuracy of computation is ensured among predicted accuracy of computing the potential energy of the second molecule; and
calculating the potential energy of the second molecule by combining the energy of each of the plurality of subsets.

7. The non-transitory computer-readable recording medium according to claim 1, wherein
the executing includes
outputting information in which each of the plurality of division candidate patterns is associated with a result of the prediction of the accuracy of computing the potential energy of the second molecule in the case of using each of the plurality of division candidate patterns.

8. A computer-implemented accuracy prediction method comprising:
generating a regression equation with which accuracy of computing potential energy of a first molecule is predicted by using each of a plurality of division patterns including a plurality of subsets each including one or more atoms included in the first molecule; and
executing prediction of accuracy of computing potential energy of a second molecule in a case of using each of a plurality of division candidate patterns including a plurality of subsets each including one or more atoms included in the second molecule by applying the regression equation to the plurality of division candidate patterns, using a processor.

9. An information processing apparatus comprising:
a processor configured to:
generate a regression equation with which accuracy of computing potential energy of a first molecule is predicted by using each of a plurality of division patterns including a plurality of subsets each including one or more atoms included in the first molecule; and
execute prediction of accuracy of computing potential energy of a second molecule in a case of using each of a plurality of division candidate patterns including a plurality of subsets each including one or more atoms included in the second molecule by applying the regression equation to the plurality of division candidate patterns.
